(19) United States
(12) Patent Application Publication
(10) Pub. No.: US 2025/0360299 A1
(43) Pub. Date: Nov. 27, 2025

(54) Torosional Non-Disconnecting Luer Connector

(71) Applicant: Becton, Dickinson and Company, Franklin Lakes, NJ (US)

(72) Inventors: Sridhaar Nandakumar, Chennai (IN); Arjun Murali, Bangalore (IN); Arun Karthik Senthil Murugan, Bangalore (IN); Suhas Hanumantharao, Bengaluru (IN); Rahul Rajan Puthukkad, Bengaluru (IN); Kowshika K, Tiruppur (IN)

(21) Appl. No.: 18/670,146

(22) Filed: May 21, 2024

(51) Int. Cl. A61M 39/10

(52) U.S. Cl. CPC A61M 39/1011; A61M 2039/1033

(57) ABSTRACT

A connector, including a connector body comprising: a first portion with a threaded surface, a second portion opposite the first portion, and a lumen extending from the first portion to the second portion; a base axially coupled to the connector body; and a torsion member coupled to the connector body and the base, wherein the torsion member is configured to permit rotation between the connector body and the base in a first direction and prevent rotation between the connector body and the base in a second direction.

### BACKGROUND OF THE INVENTION

### Field of the Invention

The present disclosure is directed to connectors for medical devices or assemblies that reduce the likelihood of inadvertent or unintentional disconnection.

### Background of the Invention

Fluid connections are routinely implemented in medical settings between components or devices involving the preparation or delivery of fluids to a patient. Such connections may include one or more Luer lock connectors to engage a syringe, vial, container, or other fluid source to an intravenous line, device, or secondary container for fluid transfer. Unintentional disconnection of a fluid connection can be problematic or harmful for both patients and clinicians. For example, unintentional disconnection of a closed system transfer device involving hazardous drugs (e.g., for chemotherapy or the like) can pose substantial health risks to a clinician. Moreover, in certain healthcare environments, such as home care settings, there may be a higher risk of unintended disconnection due children, pets, or other non-healthcare practitioners in proximity to the fluid connections of a patient. Accordingly, the present disclosure provides connectors, assemblies, and components thereof that reduce the likelihood of an unintentional disconnection of a fluid connection. The present disclosure also provides connectors, assemblies, and components thereof that may prevent disconnection altogether for single-use devices or components.

### SUMMARY OF THE INVENTION

In accordance with one aspect, the present disclosure provides a connector, comprising: a connector body comprising: a first portion with a threaded surface, a second portion opposite the first portion, and a lumen extending from the first portion to the second portion; a base axially coupled to the connector body; and a torsion member coupled to the connector body and the base, wherein the torsion member is configured to permit rotation between the connector body and the base in a first direction and prevent rotation between the connector body and the base in a second direction.

In some non-limiting embodiments or aspects, the first direction may be counter-clockwise around a longitudinal axis of the connector body.

In some non-limiting embodiments or aspects, the second direction may be clockwise around a longitudinal axis of the connector body.

In some non-limiting embodiments or aspects, the second portion of the connector body may include one or more slip surfaces, wherein each of the one or more slip surfaces may be configured to contact the torsion member to permit rotation in the first direction.

In some non-limiting embodiments or aspects, each of the one or more slip surfaces may be non-parallel to a radial axis of the connector body.

In some non-limiting embodiments or aspects, the second portion of the connector body may include one or more locking surfaces, wherein each of the one or more locking surfaces may be configured to contact the torsion member to prevent rotation in the second direction.

In some non-limiting embodiments or aspects, each of the one or more locking surfaces may be substantially parallel to a radial axis of the connector body.

In some non-limiting embodiments or aspects, the second portion of the connector body may define one or more windows in a sidewall thereof, and each window may be defined in part by at least one of the one or more locking surfaces and at least one of the one or more slip surfaces.

In some non-limiting embodiments or aspects, the torsion member may include a body and one or more arms deflectable with respect to the body.

In some non-limiting embodiments or aspects, the connector body may deflect the one or more arms to permit rotation in the first direction.

In some non-limiting embodiments or aspects, the torsion member may be axially positioned between at least a portion of the connector body and at least a portion of the base.

In some non-limiting embodiments or aspects, the torsion member may be coupled to the base such that the torsion member does not rotate with respect to the base.

In some non-limiting embodiments or aspects, at least a portion of the lumen may pass through each of the torsion member and the base.

In some non-limiting embodiments or aspects, the second portion may include a tubular member defining at least a portion of the lumen, wherein at least a portion of the tubular member may pass through an aperture in the torsion member, and wherein at least a portion of the tubular member may pass through an opening in the base.

In some non-limiting embodiments or aspects, the threaded surface may be located on an exterior of the first portion.

In accordance with one aspect, the present disclosure provides a connector, comprising: a connector body comprising: a threaded surface, a plurality of slip surfaces, a plurality of locking surfaces, and a lumen therein; a base axially coupled to the connector body; and a torsion member coupled to the connector body and the base, wherein the torsion member is configured to engage the plurality of slip surfaces to permit rotation between the connector body and the base in a first direction and engage the plurality of locking surfaces to prevent rotation between the connector body and the base in a second direction.

In some non-limiting embodiments or aspects, the first direction may be counter-clockwise around a longitudinal axis of the connector body, and/or the second direction may be clockwise around a longitudinal axis of the connector body.

In some non-limiting embodiments or aspects, the torsion member may include a body and one or more arms deflectable with respect to the body, and the connector body may radially deflect the one or more arms to permit rotation in the first direction.

In some non-limiting embodiments or aspects, each of the plurality of slip surfaces may be non-parallel to a radial axis of the connector body, and each of the plurality of locking surfaces may be parallel to the radial axis of the connector body.

In some non-limiting embodiments or aspects, at least a portion of the lumen may pass through each of the torsion member and the base.

Further examples of the present disclosure will now be described in the following numbered clauses.

Clause 1: A connector, comprising: a connector body comprising: a first portion with a threaded surface, a second portion opposite the first portion, and a lumen extending from the first portion to the second portion; a base axially coupled to the connector body; and a torsion member coupled to the connector body and the base, wherein the torsion member is configured to permit rotation between the connector body and the base in a first direction and prevent rotation between the connector body and the base in a second direction.

Clause 2: The connector of clause 1, wherein the first direction is counter-clockwise around a longitudinal axis of the connector body.

Clause 3: The connector of clause 2, wherein the second direction is clockwise around a longitudinal axis of the connector body.

Clause 4: The connector of any of clauses 1-3, wherein the second portion of the connector body includes one or more slip surfaces, wherein each of the one or more slip surfaces is configured to contact the torsion member to permit rotation in the first direction.

Clause 5: The connector of clause 4, wherein each of the one or more slip surfaces is non-parallel to a radial axis of the connector body.

Clause 6: The connector of clause 4 or clause 5, wherein the second portion of the connector body includes one or more locking surfaces, wherein each of the one or more locking surfaces is configured to contact the torsion member to prevent rotation in the second direction.

Clause 7: The connector of clause 6, wherein each of the one or more locking surfaces is substantially parallel to a radial axis of the connector body.

Clause 8: The connector of clause 6 or clause 7, wherein the second portion of the connector body defines one or more windows in a sidewall thereof, and wherein each window is defined in part by at least one of the one or more locking surfaces and at least one of the one or more slip surfaces.

Clause 9: The connector of any of clauses 1-8, wherein the torsion member includes a body and one or more arms deflectable with respect to the body.

Clause 10: The connector of clause 9, wherein the connector body deflects the one or more arms to permit rotation in the first direction.

Clause 11: The connector of any of clauses 1-10, wherein the torsion member is axially positioned between at least a portion of the connector body and at least a portion of the base.

Clause 12: The connector of any of clauses 1-11, wherein the torsion member is coupled to the base such that the torsion member does not rotate with respect to the base.

Clause 13: The connector of any of clauses 1-12, wherein at least a portion of the lumen passes through each of the torsion member and the base.

Clause 14: The connector of clause 13, wherein the second portion includes a tubular member defining at least a portion of the lumen, wherein at least a portion of the tubular member passes through an aperture in the torsion member, and wherein at least a portion of the tubular member passes through an opening in the base.

Clause 15: The connector of any of clauses 1-14, wherein the threaded surface is located on an exterior of the first portion.

Clause 16: A connector, comprising: a connector body comprising: a threaded surface, a plurality of slip surfaces, a plurality of locking surfaces, and a lumen therein; a base axially coupled to the connector body; and a torsion member coupled to the connector body and the base, wherein the torsion member is configured to engage the plurality of slip surfaces to permit rotation between the connector body and the base in a first direction and engage the plurality of locking surfaces to prevent rotation between the connector body and the base in a second direction.

Clause 17: The connector of clause 16, wherein the first direction is counter-clockwise around a longitudinal axis of the connector body, and wherein the second direction is clockwise around a longitudinal axis of the connector body.

Clause 18: The connector of clause 16 or clause 17, wherein the torsion member includes a body and one or more arms deflectable with respect to the body, and wherein the connector body radially deflects the one or more arms to permit rotation in the first direction.

Clause 19: The connector of any of clauses 16-18, wherein each of the plurality of slip surfaces is non-parallel to a radial axis of the connector body, and wherein each of the plurality of locking surfaces is parallel to the radial axis of the connector body.

Clause 20: The connector of any of clauses 16-19, wherein at least a portion of the lumen passes through each of the torsion member and the base.

### BRIEF DESCRIPTION OF THE DRAWINGS

The above-mentioned and other features and advantages of this disclosure, and the manner of attaining them, will become more apparent and the disclosure itself will be better understood by reference to the following descriptions of embodiments of the disclosure taken in conjunction with the accompanying drawings, wherein:

FIG. 1 is a perspective view of an example of a connector constructed in accordance with the principles of the present invention.

FIG. 2 is another perspective view of the connector of FIG. 1.

FIG. 3 is an exploded assembly view of the connector of FIG. 1.

FIG. 4 is a cross-sectional view of the connector of FIG. 1.

FIG. 5 is a perspective view of an example of a connector body constructed in accordance with the principles of the present invention.

FIG. 6 is another perspective view of the connector body of FIG. 5.

FIG. 7 is a perspective partial cross-sectional view of the connector body of FIG. 5.

FIG. 8 is a cross-sectional view along a lateral plane of the connector body of FIG. 5.

FIG. 9 is a perspective view of an example of a torsion member constructed in accordance with the principles of the present invention.

FIG. 10 is an opposite perspective view of the torsion member of FIG. 9.

FIG. 11 is a closer view of a portion of the torsion member of FIG. 9.

FIG. 12 is a perspective view of an example of a base constructed in accordance with the principles of the present invention.

FIG. 13 is a perspective partial cross-sectional view of the base of FIG. 12.

FIG. 14 is a side view illustrating an example of an assembly coupling a connector to a secondary connector in accordance with the principles of the present invention.

FIG. 15 is a cross-sectional view illustrating an example of an assembly coupling a connector to a secondary connector in accordance with the principles of the present invention.

FIG. 16 is a cross-sectional view along a lateral plane of the assembly of FIG. 14 in a first configuration.

FIG. 17 is a perspective cross-sectional view along of the assembly of FIG. 14 in a second configuration.

FIG. 18 is a cross-sectional view along a lateral plane of the assembly of FIG. 14 in a third configuration.

### DETAILED DESCRIPTION OF THE INVENTION

The following description is provided to enable those skilled in the art to make and use the described embodiments contemplated for carrying out the invention. Various modifications, equivalents, variations, and alternatives, however, will remain readily apparent to those skilled in the art. Any and all such modifications, variations, equivalents, and alternatives are intended to fall within the spirit and scope of the present invention.

For purposes of the description hereinafter, the terms “upper”, “lower”, “right”, “left”, “vertical”, “horizontal”, “top”, “bottom”, “lateral”, “longitudinal”, and derivatives thereof shall relate to the concept as it is oriented in the drawing figures. However, it is to be understood that the concept may assume various alternative variations, except where expressly specified to the contrary. It is also to be understood that the specific devices illustrated in the attached drawings, and described in the following specification, are simply exemplary embodiments of the concept. Hence, specific dimensions and other physical characteristics related to the embodiments disclosed herein are not to be considered as limiting.

The word “comprising” and “comprises”, and the like, does not exclude the presence of elements or steps other than those listed in any claim or the specification as a whole. In the present specification, “comprises” means “includes” and “comprising” means “including.”

As used herein, “at least one of” is synonymous with “one or more of.” For example, the phrase “at least one of A, B, or C” means any one of A, B, or C, or any combination of any two or more of A, B, or C. For example, “at least one of A, B, and C” includes A alone; or B alone; or C alone; or A and B; or A and C; or B and C; or all of A, B, and C.

The term “at least” is synonymous with “greater than or equal to.” The terms “first”, “second”, and the like are not intended to refer to any particular order or chronology, but refer to different conditions, properties, or elements. As used herein, the singular form of “a”, “an”, and “the” include plural referents unless the context clearly dictates otherwise.

All numbers used in the specification and claims are to be understood as being modified in all instances by the term “about”. By “about” is meant within plus or minus twenty-five percent of the stated value. However, this should not be considered as limiting to any analysis of the values under the doctrine of equivalents.

Unless otherwise indicated, all ranges or ratios disclosed herein are to be understood to encompass the beginning and ending values and any and all subranges or subratios subsumed therein. For example, a stated range or ratio of “1 to 10” should be considered to include any and all subranges or subratios between (and inclusive of) the minimum value of 1 and the maximum value of 10; that is, all subranges or subratios beginning with a minimum value of 1 or more and ending with a maximum value of 10 or less. The ranges and/or ratios disclosed herein represent the average values over the specified range and/or ratio.

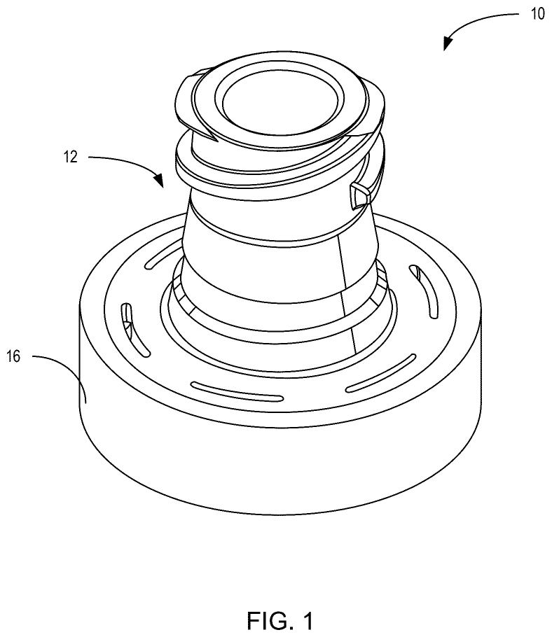

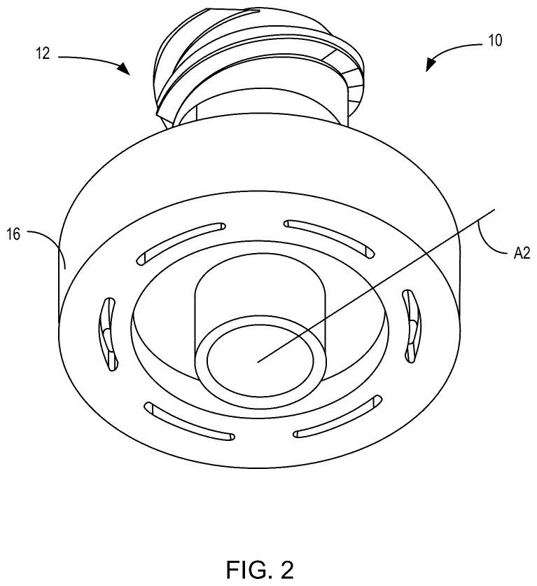

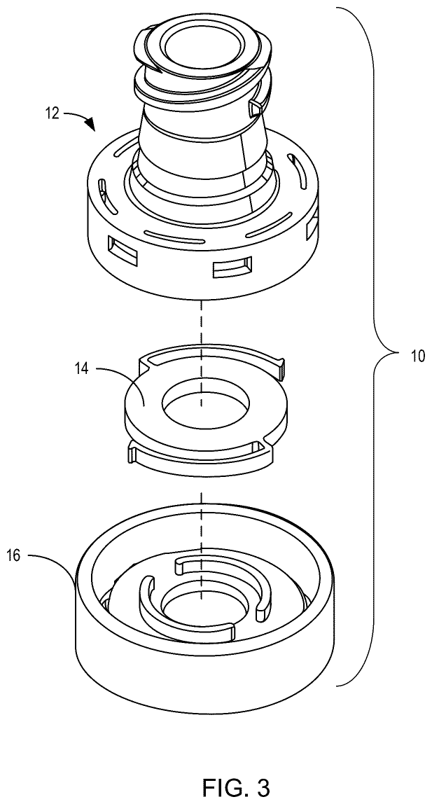

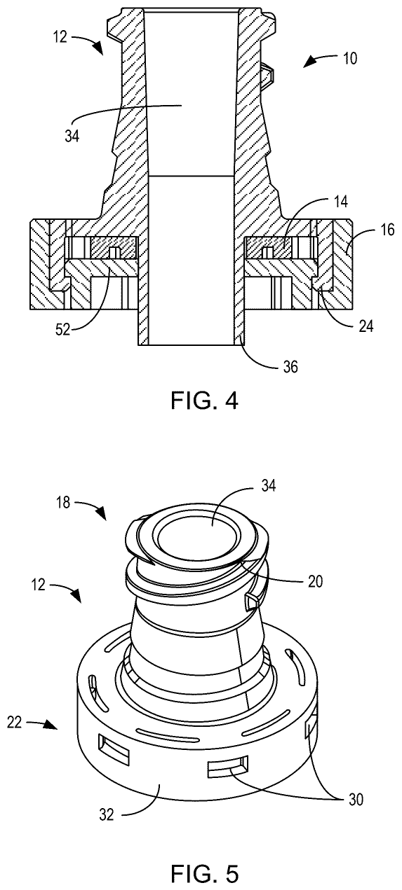

Reference is now made to FIGS. 1-4 that show an example of a connector 10 in accordance with the principles of the present disclosure 10. The connector 10 may generally include or define a connector body 12, a torsion member 14 coupled to the connector body, and a base 16 coupled to the connector body and/or torsion member 14.

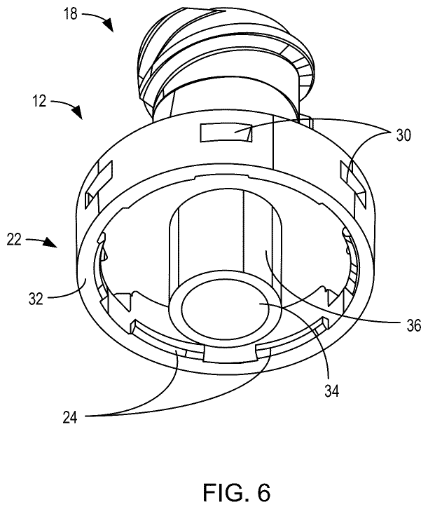

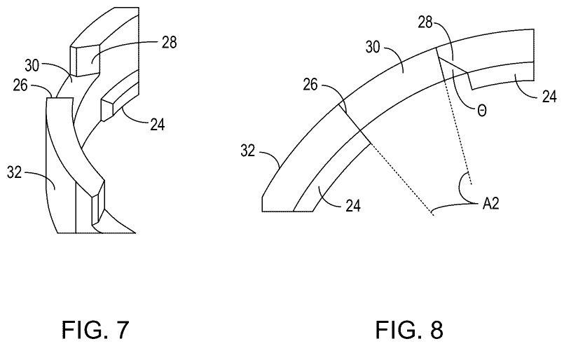

Now referring to FIGS. 5-8, the connector body 12 may generally define or include a first portion 18 configured to matably couple to or otherwise engage a complementary connector, device, or other component. For example, the first portion 18 of the connector body 12 may include or define a threaded surface 20 to threadably engage a secondary connector or device (not shown). In one example, the first portion 18 and/or the threaded surface 20 may comprise or constitute at least a portion of a female luer connector. In another example, the first portion 18 and/or the threaded surface 20 may include or constitute at least a portion of a male luer connector, or other coupling device, which may include the threaded surface 20 on an interior sidewall or other segment of the first portion 18.

The connector body 12 may generally define or include a second portion 22 opposite the first portion 18, where the second portion 22 is configured to engage the torsion member 14 and/or base 16 as described herein. In one example, the second portion 22 may include or define a substantially cylindrical portion or flange extending radially outward. The second portion 22 may include or define a diameter larger than a diameter of the first portion of the connector body 12. The second portion 22 may include or define one or more retention elements 24 configured to axially secure the connector body 12 to the torsion member 14 and/or base 16. For example, the retention elements 24 may include a plurality of snaps or tabs radially spaced around a circumference or perimeter of the second portion 22 of the connector body 12 that engage one or more portions or features of the torsion member 14 and/or base 16 to prevent or restrict axial movement between the connector body 12 and the torsion member 14 and/or base 16. In one example, the retention elements 24 prevent or restrict axial movement between the connector body 12 and the torsion member 14 and/or base 16 while allowing rotational movement between the connector body 12 and at least one of the torsion member 14 and/or base 16.

The connector body 12 may be configured to permissibly rotate in a first direction with respect to the torsion member 14 and/or base 16, while being restricted or prevented from rotating in a second, opposite direction with respect to the torsion member 14 and/or base 16. The connector body 12 may include or define one or more structures and/or surfaces on the first and/or second portions 18, 22 to provide such rotational characteristics. For example, the connector body 12 may include or define one or more locking surfaces 26 configured to abut, engage, or otherwise contact a portion of the torsion member 14 that, once contacted, restricts or prevents further rotation of the connector body 12 with respect to the base 16 and/or the torsion member 14. The one or more locking surfaces 26 may be on, in, or otherwise coupled to the second portion 22 of the connector body 12. The one or more locking surfaces 26 may include a plurality of locking surfaces 26 radially spaced equidistant around a circumference and/or perimeter of the second portion 22 or other segment of the connector body 12. The one or more locking surfaces may include a substantially planar face or surface that is substantially parallel to a longitudinal axis A1 (e.g., a substantially ‘vertical’ surface when the first portion 18 is considered the ‘top’ of the connector body), and/or substantially parallel to a radial axis or centerline A2 of the connector body 12.

The connector body 12 may include or define one or more slip surfaces 28 configured to abut, engage, or otherwise contact a portion of the torsion member 14 and, once contacted, permit further or continued rotation of the connector body 12 with respect to the base 16 and/or the torsion member 14. The one or more slip surfaces 28 may be on, in, or otherwise coupled to the second portion 22 of the connector body 12. The one or more slip surfaces 28 may include a plurality of slip surfaces radially spaced equidistant around a circumference and/or perimeter of the second portion 22 or other segment of the connector body 12. The one or more slip surfaces 28 may include a substantially planar face or surface that is substantially parallel to the longitudinal axis A1 (e.g., a substantially ‘vertical’ surface when the first portion 18 is considered the ‘top’ of the connector body), and/or substantially non-parallel or otherwise forming an angle θ with respect to the radial axis or centerline A2 of the connector body 12. In one example, the angle θ may be between 45 degrees and 55 degrees.

The connector body 12 may include or define one or more windows 30 located on or about the second portion 22. For example, the one or more windows 30 may include a plurality of windows radially spaced around and extending through a sidewall 32 on, in, or constituting a portion of the second portion 22 of the connector body. Each of the one or more windows 30 may be formed or defined at least in part by one of the locking surfaces 26 and/or one of the slip surfaces 28. For example, one of the locking surfaces 26 may form a first sidewall or lateral end of one of the windows 30, while one of the slip surfaces 28 may form a second, opposite sidewall or lateral end of the window 30.

The connector body 12 may include or define a lumen 34 therein extending from the first portion 18 to the second portion 22. The lumen 34 may be configured to provide fluid communication through the connector body 12 to render the connector 10 a fluid coupling. The lumen 34 may extend along and/or coaxial with the longitudinal axis A1. The connector body 12 may include or define a tubular portion 36 extending through, below, and/or from the second portion 22 that includes or defines a segment of the lumen 34. The tubular portion 36 may be configured for insertion into or integration with a fluid line, hub, or other portion of a fluid delivery or circulation system.

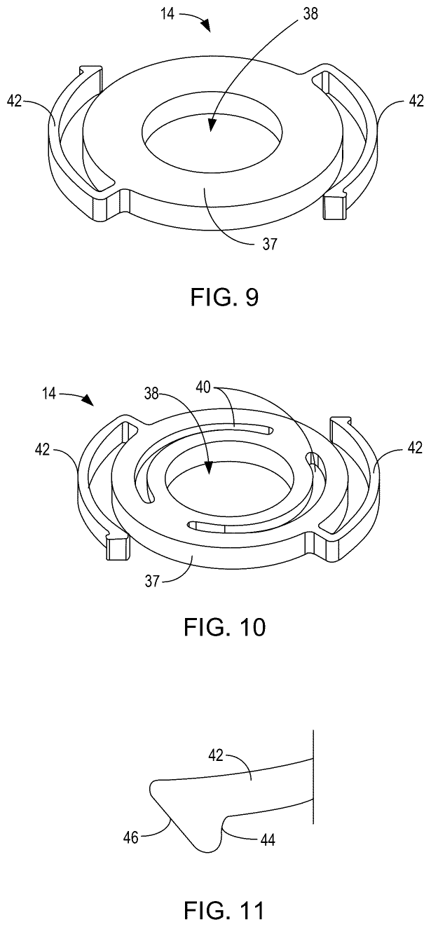

Now referring to FIGS. 9-11, an example of the torsion member 14 is shown. The torsion member 14 may include or define a body 37 positionable between at least a portion of the connector body 12 and the base 16. The body 37 may be plate-like or low profile comparative to the height and/or width of the connector body 12 and/or the base 16. The body 37 may be annular or ring-like. The torsion member 14 may be configured to be coaxially aligned with the connector body 12 and the base 16 along the longitudinal axis A1. The body 37 of the torsion member may 14 include or define an aperture 38 therethrough configured to receive at least a portion of the connector body 12, such as the second portion 22 and/or the tubular portion 36 for example. The torsion member 14 may include or define one or more features that matably engage or couple to complementary features on the base 16 that prevent rotation between the torsion member 14 and the base 16. For example, the body 37 of the torsion member 14 may include one or more grooves 40 on an underside or surface facing the base 16 that receive a portion of the base 16, as described herein, to prevent rotation between these components. Additional and/or alternative affixation mechanisms or structures may be implemented to provide a rotationally-restrictive engagement between the torsion member 14 and the base 16.

The torsion member 14 may be configured to permit rotation between the connector body 12 and the base 16 in a first direction, while restricting or preventing rotation between the connector body 12 and the base 16 in a second direction. In one example, the torsion member 14 may permit rotation between the connector body 12 and the base 16 around the longitudinal axis A1 in a counter-clockwise direction, and restrict or prevent rotation between the connector body 12 and the base 16 around the longitudinal axis A1 in a clockwise direction. In one example, the torsion member 14 may include one or more arms 42 extending or included on or about the body 37 of the torsion member 14. The arms 42 may include a plurality of arms radially spaced around a circumference or perimeter of the body 37 of the torsion member 14. In one example, the one or more arms 42 may include first and second arms spaced approximately 180 degrees away from each other. Each of the one or more arms 42 may form, define, or include a cantilever joint and/or living hinge with respect to the body 37 of the torsion member 14 such that at least a portion of each of the one or more arms 42 is deflectable and/or movable with respect to the body 37 of the torsion member 14. In one example, each of the one or more arms 42 may be radially deflectable towards and/or away from the body 37 and/or a center point thereof.

Each of the one or more arms 42 may include or define one or more surfaces configured to engage, abut, or otherwise contact the connector body 12 to provide the rotational features described herein. For example, each of the one or more arms 42 may include a first surface 44 configured to engage, abut, or otherwise contact the one or more locking surfaces 26 to prevent rotation between the connector body 12 and the base 16 and/or torsion member 14 in a first direction. The first surface 44 may be configured and/or oriented such that it is substantially parallel to the locking surface 26 when in contact with the locking surface 26.

Each of the one or more arms 42 may include a second surface 46 configured to engage, abut, or otherwise contact the one or more slip surfaces 28 to permit rotation between the connector body 12 and the base 16 and/or torsion member 14 in a second direction. The second surface 46 may be configured and/or oriented such that it is substantially parallel to the slip surface 28 when in contact with the slip surface 28.

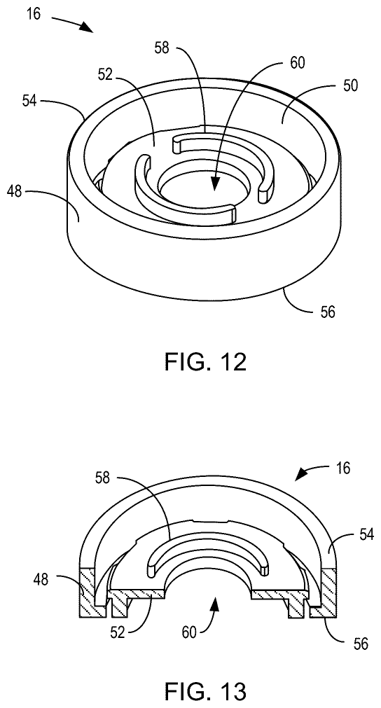

Now referring to FIGS. 12-13, an example of the base 16 is shown. The base 16 may generally be configured to axially receive and/or couple to the torsion member 14 and the connector body 12. The base 16 may include or define a body 48 sized and shaped to receive at least a portion of each of the torsion member 14 and the connector body 12 therein. For example, the body 48 of the base may be substantially cylindrical and include or define an interior cavity 50 therein. The body 48 may include or define a platform 52 positioned within the cavity 50, where the platform 52 is axially spaced from both an upper, top edge or end 54 of the body 48 and a lower, bottom edge or end 56 of the body 48. The platform 52 may be substantially circular in shape and have a dimeter or width less than an overall diameter or width of the body 48 of the base 16. The platform 52 and/or other portion of the body 48 of the base 16 may include or define a flange, shelf, edge, or other structure to facilitate axial coupling of the connector body 12 to the base 16. For example, one or more segments of the perimeter or outer circumference of the platform may be sized and shaped to receive and anchor the one or more retention elements 24 of the connector body 12. The coupling between the one or more retention elements 24 of the connector body 12 and the base 16 may restrain or prevent axial movement therebetween, but provide uninhibited rotation about the longitudinal axis A1 between the connector body 12 and the base 16.

The base 16 may include or define one or more features that matably engage or couple to complementary features on the torsion member 14 that prevent rotation between the torsion member 14 and the base 16. For example, the platform 52 may include one or more protrusions 58 that engage the groves 40 of the torsion member 14 when the torsion member 14 is axially posited within and coupled to the base 16. Additional and/or alternative affixation mechanisms or structures may be implemented on the base 16 to provide a rotationally-restrictive engagement between the torsion member 14 and the base 16.

The base 16 may include or define an opening 60 therein to receive at last a portion of the connector body 12 therethrough. For example, the opening 60 may be in the platform 52 to permit the tubular portion 36 of the connector body 12 to pass through and extend below the bottom 56 of the body 48 of the base 16.

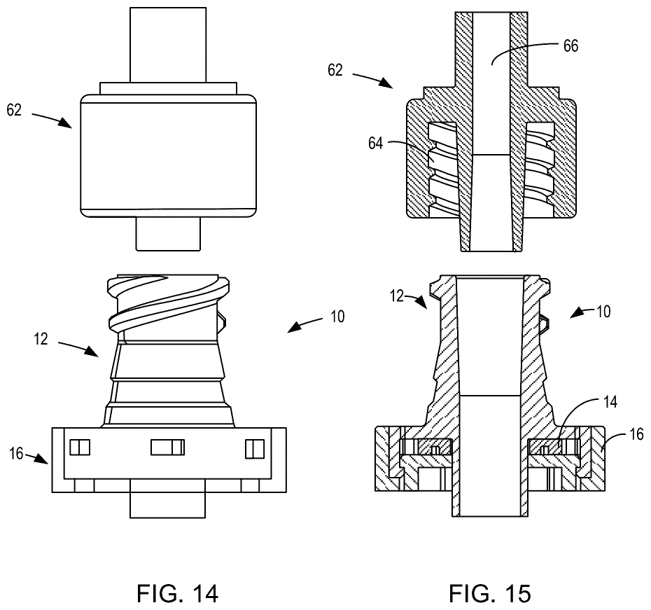

Now referring to FIGS. 14-18, an example of use of the connector 10 with a secondary connector 62 to provide a secure fluid connection is shown. The secondary connector 62 may generally include or define a threaded surface 64 configured to engage the threaded surface 20 of the connector body 12. The secondary connector 62 may include or define a fluid channel 66 configured to engage and provide fluid communication with the lumen 34 of the connector body 12. The secondary connector 62 may include or be part of, for example, a male luer lock connector, a hub, a container or syringe, a fluid line, or any other component of a fluid delivery, transfer, or storage system.

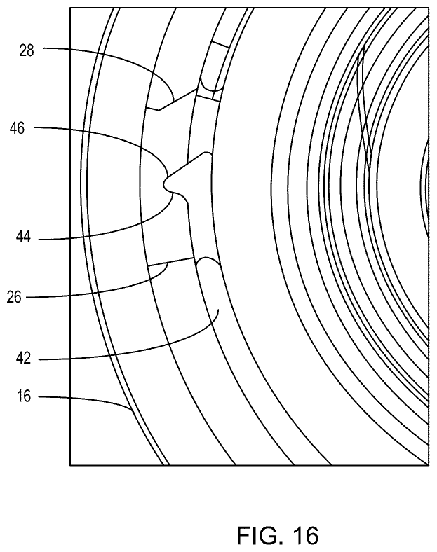

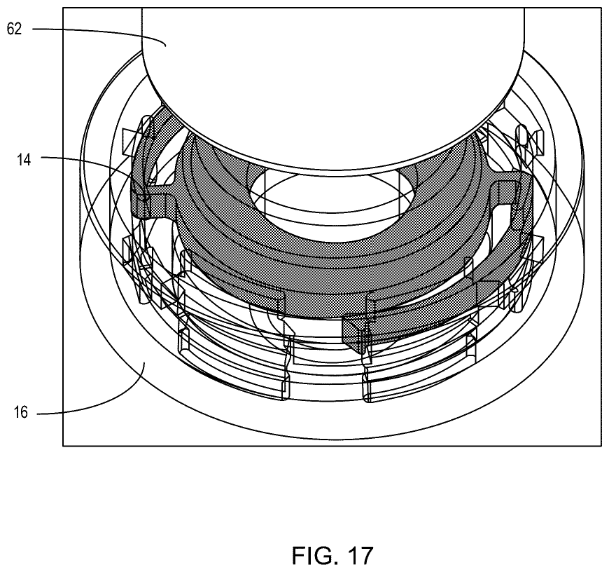

In one example of use, the secondary connector 62 may be axially aligned with the connector 10 and advanced axially while rotating to couple the threaded surface 64 of the secondary connector 62 with the threaded surface 20 of the connector body 12. The axial and rotational advancement positions a portion of the fluid channel 66 into the lumen 34 of the connector body. In illustrated example, the secondary connector 62 is rotated clockwise to engage the threads. As shown in FIG. 16, an initial position of an end of the one or more arms 42 of the torsion member 14 may be in between a locking surface 26 and a slip surface 28 of the connector body 12. As the secondary connector 62 is tightened to the connector body, the rotational torque of the secondary connector 62 is transmitted to the connector body 12, which would cause the connector body 12 to rotate clockwise with respect to the torsion member 14 and the base 16. The rotation of the connector body 16 may continue until one or more of the locking surfaces 26 engages the first surface 44 of the arms 42 of the torsion member 14, as shown in FIG. 17. This engagement between the torsion member 14 and the connector body 16 prevents further clockwise rotation of the connector body 16 with respect to the base 16, and prevents the connector body 16 from rotating clockwise in unison with the secondary connector 62, thereby allowing the secondary connector 62 to be tightened and sealed to the connector body 16 to a desirable torque limit or specification.

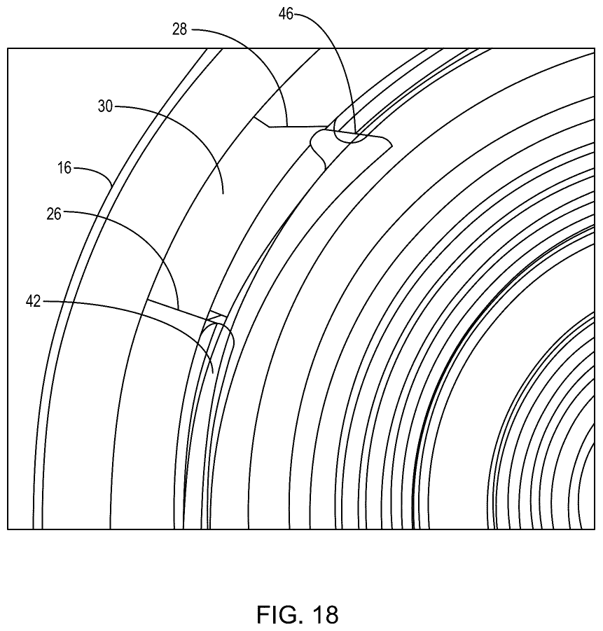

Attempting to remove the secondary connector 62 from the connector 10 would typically include rotating the secondary connector 62 in an opposite direction with respect to the connector 10 to unthread and disconnect the threaded surface 64 of the secondary connector 62 with the threaded surface 20 of the connector body 12. However, rotating the secondary connector 62 counter clockwise would rotate the connector body 12 with respect to the base 16 and torsion member 14. The counterclockwise rotation would result in one or more of the slip surfaces 28 engaging or contacting the second surface 46 of the one or more arms 42 of the torsion member, as shown in FIG. 18. This engagement or contact results in radial deflection of the one or more arms 42, which permits the connector body 12 to continue rotating, in unison, with the secondary connector and with respect to the base 16 and torsion member 14. Accordingly, the secondary connector 62 cannot be disconnected from connector body 12 and the connector 10.

While the examples disclosed herein illustrate thread pitches involving clockwise movement for tightening or connecting the components, and counterclockwise movement for disconnecting or loosening the components, it is contemplated that the thread pitch may be reversed in some environments or applications, resulting in opposite rotational movements to those described herein to achieve or enable the features and benefits disclosed herein.

It will be appreciated by persons skilled in the art that the present disclosure is not limited to what has been particularly shown and described herein above. In addition, unless mention was made above to the contrary, it should be noted that all of the accompanying drawings are not to scale. Of note, the system components have been represented where appropriate by conventional symbols in the drawings, showing only those specific details that are pertinent to understanding the embodiments of the present disclosure so as not to obscure the disclosure with details that will be readily apparent to those of ordinary skill in the art having the benefit of the description herein. Moreover, while certain embodiments or figures described herein may illustrate features not expressly indicated on other figures or embodiments, it is understood that the features and components of the examples disclosed herein are not necessarily exclusive of each other and may be included in a variety of different combinations or configurations without departing from the scope and spirit of the disclosure. A variety of modifications and variations are possible in light of the above teachings without departing from the scope and spirit of the disclosure, which is limited only by the following claims.

What is claimed is:

1. A connector, comprising:
a connector body comprising:
a first portion with a threaded surface,
a second portion opposite the first portion, and
a lumen extending from the first portion to the second portion;
a base axially coupled to the connector body; and
a torsion member coupled to the connector body and the base, wherein the torsion member is configured to permit rotation between the connector body and the base in a first direction and prevent rotation between the connector body and the base in a second direction.

2. The connector of claim 1, wherein the first direction is counter-clockwise around a longitudinal axis of the connector body.

3. The connector of claim 2, wherein the second direction is clockwise around a longitudinal axis of the connector body.

4. The connector of claim 1, wherein the second portion of the connector body includes one or more slip surfaces, wherein each of the one or more slip surfaces is configured to contact the torsion member to permit rotation in the first direction.

5. The connector of claim 4, wherein each of the one or more slip surfaces is non-parallel to a radial axis of the connector body.

6. The connector of claim 4, wherein the second portion of the connector body includes one or more locking surfaces, wherein each of the one or more locking surfaces is configured to contact the torsion member to prevent rotation in the second direction.

7. The connector of claim 6, wherein each of the one or more locking surfaces is substantially parallel to a radial axis of the connector body.

8. The connector of claim 6, wherein the second portion of the connector body defines one or more windows in a sidewall thereof, and wherein each window is defined in part by at least one of the one or more locking surfaces and at least one of the one or more slip surfaces.

9. The connector of claim 1, wherein the torsion member includes a body and one or more arms deflectable with respect to the body.

10. The connector of claim 9, wherein the connector body deflects the one or more arms to permit rotation in the first direction.

11. The connector of claim 1, wherein the torsion member is axially positioned between at least a portion of the connector body and at least a portion of the base.

12. The connector of claim 1, wherein the torsion member is coupled to the base such that the torsion member does not rotate with respect to the base.

13. The connector of claim 1, wherein at least a portion of the lumen passes through each of the torsion member and the base.

14. The connector of claim 13, wherein the second portion includes a tubular member defining at least a portion of the lumen, wherein at least a portion of the tubular member passes through an aperture in the torsion member, and wherein at least a portion of the tubular member passes through an opening in the base.

15. The connector of claim 1, wherein the threaded surface is located on an exterior of the first portion.

16. A connector, comprising:
a connector body comprising:
a threaded surface,
a plurality of slip surfaces,
a plurality of locking surfaces, and
a lumen therein;
a base axially coupled to the connector body; and
a torsion member coupled to the connector body and the base, wherein the torsion member is configured to engage the plurality of slip surfaces to permit rotation between the connector body and the base in a first direction and engage the plurality of locking surfaces to prevent rotation between the connector body and the base in a second direction.

17. The connector of claim 16, wherein the first direction is counter-clockwise around a longitudinal axis of the connector body, and wherein the second direction is clockwise around a longitudinal axis of the connector body.

18. The connector of claim 16, wherein the torsion member includes a body and one or more arms deflectable with respect to the body, and wherein the connector body radially deflects the one or more arms to permit rotation in the first direction.

19. The connector of claim 16, wherein each of the plurality of slip surfaces is non-parallel to a radial axis of the connector body, and wherein each of the plurality of locking surfaces is parallel to the radial axis of the connector body.

20. The connector of claim 16, wherein at least a portion of the lumen passes through each of the torsion member and the base.
